(19) United States
(12) Patent Application Publication
(10) Pub. No.: US 2017/0007570 A1
(43) Pub. Date: Jan. 12, 2017

(54) PREPARATIONS OF TAXANES FOR INTRAVENOUS ADMINISTRATION AND THE PREPARATION METHOD THEREOF

(71) Applicant: TASLY HOLDING GROUP CO., LTD., TIANJIN (CN)

(72) Inventors: Jianming Chen, TIANJIN (CN); Baoan Gao, TIANJIN (CN); Jing Sun, TIANJIN (CN); Yue Zhang, TIANJIN (CN); Xiaoli Zheng, TIANJIN (CN); Ying Li, TIANJIN (CN); Dan Guo, TIANJIN (CN); Yang Zhang, TIANJIN (CN); Zhongbin Wu, TIANJIN (CN); Qiuxia Yang, TIANJIN (CN); Wei Liu, TIANJIN (CN); Peng Gu, TIANJIN (CN)

(73) Assignee: TASLY HOLDING GROUP CO., LTD., TIANJIN (CN)

(21) Appl. No.: 15/274,452

(22) Filed: Sep. 23, 2016

Related U.S. Application Data
(63) Continuation of application No. 14/059,347, filed on Oct. 21, 2013.
Application No. 14/059,347 is a continuation of application No. 12/571,176, filed on Sep. 30, 2009, now abandoned.

(51) Int. Cl. A61K 31/337; A61K 47/12; A61K 9/107; A61K 47/02; A61K 9/00; A61K 47/10

(52) U.S. Cl. CPC A61K 31/337; A61K 9/0019; A61K 47/10; A61K 9/107; A61K 47/02; A61K 47/12

(57) ABSTRACT

The invention relates to a preparation of taxanes for intravenous administration consisting of a drug solution and an emulsion. The drug solution consists of paclitaxel or docetaxel, a pH regulator and an organic solvent for injection. The emulsion includes a fat emulsion and is composed of oil for injection, an emulsifier, an antioxidant, an isotonic regulator, a stabilizer, a pH regulator and water for injection. When used, the drug solution at the clinical dosage can be added and evenly mixed in the emulsion to perform intravenous drip directly; or the drug solution at the clinical dosage can also be firstly added into the emulsion with no less than 5 times volume of the drug solution and then a predetermined amount of normal saline or glucose solution for injection is added to perform intravenous drip. The preparation of the present invention does not contain solubilizer.

### FIELD OF THE INVENTION

The present invention relates to the field of medical technology. More specifically, the present invention relates to the preparations of taxanes for intravenous administration and the preparation method thereof.

### BACKGROUND OF THE INVENTION

Paclitaxel (Taxol™) and docetaxel (Taxotem™) are two types of taxane anticancer drugs approved by Food & Drug Administration (FDA), wherein the paclitaxel, as a natural product, is an anticancer chemical ingredient extracted from the bark of the mountain mahogany (Taxus brevifolia Nutt.) tree, while the docetaxel is a product semi-synthesized from the precursor extracted from the needle leaves of the Taxus baccata tree.

The taxanes belong to a type of typical cytotoxic agents with a wide spectrum of anticancer effect, having strong inhibitory effect on both primary and metastatic tumors such as breast cancer, ovarian cancer, non small lung cancer (NSCLC), head and neck squamous cell carcinoma and malignant melanoma. Its anti-tumor mechanism lies in that promoting the assembly of tubulin dimer into microtubule and further making the microtubule super-stable by stimulating polymerization of tubulin, thus inhibiting the microtubule net from kinetic recombination. Consequently, the proliferation of cancer cell is prevented at the resting stage of mitosis, and thus achieves the purpose of an anti-cancer effect.

Due to the poor water-solubility and oil-solubility of paclitaxel and docetaxel, they can be hardly dissolved in water (4 μg/ml of water-solubility) and only 2%-4% is absorbed after oral administration. On the other hand, paclitaxel and docetaxel can be dissolved in an organic solvent such as anhydrous ethanol, but when normal saline is added, the drug is precipitated immediately from the drug solution prepared with the organic solvent. This makes the intravenous drip impossible in clinical application. Hence, polyoxyethylated castor oil (Cremophor® EL), is usually used as a solubilizer in the available paclitaxel injection preparation. In the paclitaxel injection preparation, the drug solution of paclitaxel is a colorless viscous concentrated solution prepared by a mixed solvent of Cremophor® EL and anhydrous ethanol in the ratio of 50:50 (v/v). Because of the presence of the solubilizer, the drug is not precipitated immediately when normal saline is added in clinical practice. However, the preparation has a short stability time, which makes it necessary to complete the intravenous drip in a short period of time; otherwise, the drug will be precipitated. But such quick infusion might cause certain risk to some patents, which is one of drawbacks for the preparation. The second drawback is the serious toxic and side effects induced by the solubilizer, the Cremophor® EL. The acute and common clinical side effects of the available paclitaxel preparation after administration are severe: dyspnea, flushing face, palpitation and allergic reaction such as skin rash etc, which brings a lot of potential safety troubles and suffering to the patients. As a result, pre-administration of anti-allergy drug is a feasible way usually adopted to alleviate the side effects, so the available preparation is not an ideal one.

Similarly, there are problems in available docetaxel preparation. The solvent of the drug solution is composed of a solution of Tween 80 and 13% ethanol solution, in which the Tween 80, utilized as a solubilizer, has an effect for hemolysis to some degree. In addition, the addition of normal saline when administrating it to the patients will also have the stability time shortened, so it is needed to complete the intravenous drip in a short period of time. Therefore, the available docetaxel preparation has less drug safety in the clinical application.

Now, a lot of research works on the paclitaxel emulsion-related preparation have been reported. For example, Kan et al. had developed a paclitaxel O/W emulsion by nonionic surfactant and phospholipid (Kan P. et al., Controlled Release, 1999, 58: 271-278). However, Tween 80 is contained in this preparation, which leads to hemolysis in patients after intravenous drip, causing serious side effects. In the application entitled “Parenteral paclitaxel in a stable non-toxic preparation” filed by B. S. Anderson (Chinese Appl. No.: 97196934,5), dimethylacetamide and PEG were used in the composition of the parenteral preparation, in which dimethylacetamide, utilized as a solubilizer, had effects for inducing toxicity and hemolysis to some degree.

### SUMMARY OF THE INVENTION

The present invention provides solubilizer-free, less toxic and more stable preparations of taxanes for intravenous administration.

The preparations of taxanes for intravenous administration of the present invention consist of two parts: a drug solution containing paclitaxel or docetaxel, and an emulsion. The solvent of said drug solution is an organic solvent; and the emulsion includes a fat emulsion. When used, the drug solution at the clinical dosage can be added and mixed homogeneously in the emulsion to perform intravenous drip directly; or the drug solution at the clinical dosage can also be firstly added into the emulsion with no less than 5 times volume of the drug solution and then a predetermined amount of normal saline or glucose solution for injection is added to perform the intravenous drip.

It is well-known that the fat emulsion belongs to one kind of emulsion, and the emulsion consists of an oil phase and a water phase. The structure of the emulsion micro-particles can be divided into two parts, an inner core and an outside layer, the former is made up of low polar oils and hydrophobic groups of surfactant to form a non-polar hydrophobic area; and the latter is made up of polar groups of the hydrated surfactant. Furthermore, a transitional layer with increasing polarity from the inner core to the outside layer is formed in the structure. According to the theory of similarity and intermiscibility, drugs with different polarity can usually find their corresponding polar areas in this transitional polar environment of the emulsion micro-particles; hence the emulsion can be used as a drug carrier. Considering the poor water and oil solubility demonstrated by paclitaxel and docetaxel, after the drug solution is dissolved in the emulsion solution, some of the emulsion micro-particles are used as the carrier carry the drug, and then these drug-loaded emulsion micro-particles are further dispersed homogeneously into other drug-unloaded emulsion to form a stable preparation. This is one of reasons for using an emulsion in the present invention. In addition, the emulsion has a targeting effect, which can deliver drug to the tumor or other lesion site. Further, the fat emulsion can be used as a nutritional agent. Clinically, it is usually administrated intravenously to the patient in need of high calorie intake, such as the patients with tumor and other malignant diseases, the protein-forbidden patients due to renal injury as well as the patients unable to uptake nutrition via gastrointestinal tract for some reason. It is remarkable that, compared with long chain fatty glycerides such as soybean oil, medium chain fatty glycerides represented by octyl and decyl glycerate display better solubility, absorption, high compatibility and anti-oxidation. This is another reason for using an emulsion including fat emulsions prepared. by long and medium chain triglycerides in the present invention.

As shown in the experiment, the preparations of taxanes for ravenous administration of the present invention completely comply with the requirements for clinical application. It offers some advantages as follows:

(1) The preparations of the present invention have excellent safety. The preparations of the present invention do not contain any solubilizer such as dimethylacetamide and Tween-80 etc., thus the toxicity and side effect are reduced accordingly. The adjuvants used have good biocompatibility and tolerance in vivo.

(2) The preparations of the present invention have high stability. Within 48 hours after mixing paclitaxel or docetaxel solution with emulsion or fat emulsion, no obvious precipitation or degradation of drug is observed. Even if the addition of normal saline or glucose solution for injection is followed, many pharmaceutical parameters such as the drug content, particle size of the emulsion and pH value do not change significantly, which completely meets the clinical needs, hence solving the problem of immediate precipitation of drugs caused by dispersion of drug into the normal saline or glucose solution for injection.

(3) The preparations of the present invention can be used as nutritional agents. Not only does a fat emulsion have the targeting effect when used as a drug carrier, but can provide nutritional replenishment for the tumor patient, hence, achieving a better therapeutic effect.

(4) The preparations of the present invention are cost-efficient and convenient for transportation and storage in practice. The reason is that the normal saline or glucose solution for injection can be used to replace a considerable proportion of the emulsion, thus the amount of emulsion or fat emulsion is reduced.

The preparations of taxanes for intravenous administration of the present invention are composed of two parts, a drug solution and an emulsion. The ingredients and proportions are as follows:

Drug solution

| Ingredients | Content % (w/v) |
|---|---|
| Paclitaxel or docetaxel | 0.01-10 |
| pH regulator | A proper amount to adjust pH value to 4.0-7.0 |
| Solvent for injection | Balanced (Please note: “balanced” |
| | herein means that t [symbol] |
| | residual amount except for paclitaxel |
| | or docetaxel and [symbol] |
| | regulator in the drug solution.) |

[symbol]

indicates data missing or illegible when filed

Wherein, the solvent for injection is an organic solvent, including one or more kinds selected from a group consisting of PEG (polyethylene glycol)-200, PEG-300, PEG-400, PEG-600, propylene glycol, glycerol and anhydrous ethanol, optionally comprising water for injection with an amount of no more than 50% of total amount of the drug solution. The pH regulator is one or more kinds selected from a group consisting of citric acid, malic acid, hydrochloric acid, acetic acid, sodium carbonate, sodium bicarbonate and sodium hydroxide.

Emulsion

| Ingredients | Content % (w/v) |
|---|---|
| Oil for injection | 1-50 |
| Emulsifier | 0.5-10 |
| Antioxidant | 0-0.5 |
| Isotonic regulator | A proper amount adjusted to the osmotic |
| | pressure in the human body |
| Stabilizer | 0-5 |
| pH regulator | A proper amount to adjust pH value to 4.0-9.0 |
| Water for injection | Balanced (Please note: “balanced” herein |
| | means that the resid [symbol] |
| | amount except for oil for injection, |
| | emulsifier, antioxida [symbol] isotonic regulator, stabilizer, |
| | and pH regulator in the emulsion.) |

[symbol]

indicates data missing or illegible when filed

Wherein, the oil for injection may be one or more oils selected from a group consisting of octyl and decyl glycerate, monooctanoin, dicaprylin, trioctanoin, Ganoderma lucidum spores oil, monodecanoin, didecanoin, tridecanoin, octyl and decyl monoglyceride, coix seed oil, Brucea Javanica oil, Herba Artemisiae Annuae oil, octyl and decyl diglyceride, soybean oil, fish oil, linseed oil, helianthus annuus seed oil, evening primrose oil, sea buckthorn oil, zedoary turmeric oil, safflower seed oil, sesame oil, corn oil, elemene oil and stearic acid. The emulsifier may be one or more emulsifiers selected from a group consisting of soybean phospholipid, yolk phospholipid, cholesterol, poloxamer 188 and glyceryl monooleate. The antioxidant may be tocopherol. The isotonic regulator may be one or more isotonic regulators selected from a group consisting of glycerol, sorbitol, mannitol, glucose and sodium chloride to adjust the osmotic pressure to that in human body. The stabilizer may be one or more stabilizers selected from a group consisting of oleic acid, sodium oleate, cholic acid and sodium cholate. The pH regulator may be one or more stabilizers selected from a group consisting of citric acid, malic acid, hydrochloric acid, acetic acid, sodium carbonate, sodium bicarbonate and sodium hydroxide.

According to the present invention, a method to prepare the preparations of taxanes for intravenous administration is described as follows:

a) Preparing a drug solution:

paclitaxel or docetaxel is added to a solvent for injection in a predetermined proportion and stirred at 50-100° C. to dissolve. The pH value of the obtained solution was adjusted to 4.0-7.0 by using a pH regulator, and 0.01%-5% (W/V) activated carbon for injection use is added to perform adsorption for 15-120 min at 25-100° C. Next, the solution is filtrated, separately packaged, sterilized and packaged using routine methods to obtain the drug solution. The obtained solution is a transparent liquid.

b) Preparing an Emulsion:

b-1) Preparing an Oil Phase: an emulsifier or stabilizer is added into an oil injection in a predetermined proportion, stirred at 50-90° C. to dissolve, into which tocopherol is added and dissolved by stirring or ultrasonicating to obtain the oil phase;

b-2) Preparing a Water Phase: the emulsifier or stabilizer, and isotonic regulator are added into water for injection in a predetermined proportion, stirred. at 50-90° C. to dissolve to obtain the water phase;

While preparing the oil phase or water phase, said emulsifier an stabilizer may be added simultaneously or separately.

b-3) Preparing the Emulsion: the oil phase of step b-1) is mixed with the water phase of step b-2) at 50-90° C., and followed by emulsification by use of a shear emulsifying machine or stirring emulsification for 5-300 min at a rotation speed of 300-8000 rpm to obtain an initial emulsion, and the pH value of the initial emulsion is adjusted by the pH regulator to 4.0-9.0. The obtained initial emulsion is further emulsified and diluted to volume with water for injection, filtrated, separately packaged, charged with nitrogen and sterilized by routine method, thus the emulsion is obtained. The final emulsion has the appearance of a white or off-white colored emulsified liquid with opalescence, and the particle size of the emulsion micro-particles ranges from 50-500 nm.

Wherein, in the above step of further emulsifying the initial emulsion, emulsifying methods used in the present invention include but are not limited to emulsification by high-pressure homogenizer, mechanical stirring, ultrasound or colloid mill. The preferred method is emulsification by high-pressure homogenizer under a pressure of 5000-25000 psi. In the step of preparing the drug solution and emulsifier, sterilization methods used in the present invention include but are not limited to using rotary high-pressure steam sterilizer, circulating vapor or micro-porous filter membrane etc. The preferred method is using rotary high-pressure steam sterilizer carried out at 100-121° C. for 20-60 min. The filtrating equipment includes but is not limited to micro-porous filter membrane, sand filtrating bar, sintered filter funnel or bladder-type filter etc.

According to the present invention, the preparations of taxanes for intravenous administration can be administered in two ways: the drug solution at the clinical dosage can be added and mixed homogeneously in the emulsion to perform the intravenous drip directly; or the drug solution at the clinical dosage can also be firstly added into the emulsion with no less than 5 times volume of the drug solution and then a predetermined amount of normal saline or glucose solution for injection is added to perform intravenous drip.

According to the present invention, the preparations of taxanes for intravenous administration are solubilizer-free and have advantages of safety, effectiveness, stability and economy. The fat emulsion is also used as a nutritional replenishment for the patients, thus achieving a better therapeutic effect. In addition, the normal saline or glucose solution for injection can be used to replace a considerable proportion of the emulsion, which makes the preparations of the present invention more cost-efficient and convenient for transportation and storage in practice. The present invention also can be used to prepare the preparations of other poorly water or oil soluble medicinal compounds for intravenous administration.

### BEST MODES OF THE INVENTION

The following examples are described to demonstrate preferred embodiments of the present invention.

### EXAMPLE 1

### Preparing Paclitaxel Preparation for Intravenous Administration

a) Preparing the Drug Solution: 2.5 g paclitaxel was added to 100 ml PEG-400, and stirred at 70° C. to dissolve the paclitaxel. The pH value of the solution was adjusted to 5.5 by using hydrochloric acid and sodium bicarbonate, and 0.2 g activated carbon for injection use was added to perform adsorption at 25° C. for 30 min. Next, the solution was filtrated by 0.45 μm micro-porous filter membrane, separately packaged, sterilized by high-pressure steam at 115° C. for 30 min, and the drug solution was thus obtained;

b) Preparing the Emulsion

b-1) Preparing the Oil Phase: 200 g octyl and decyl glycerate for injection was heated to 70° C. in a water bath, into which 12 g soybean phospholipid for injection was added to dissolve by stirring, and then 0.5 g tocopherol was added and stirred well to obtain the oil phase;

b-2) Preparing the Water Phase: glycerol and 10 g poloxamer 188 were added into 640 ml water for injection, and stirred at 70° C. to dissolve the glycerol and poloxamer to obtain the water phase;

b-3) Preparing the Emulsion: the oil phase was mixed with the water phase at 70° C., and followed by emulsification using shear emulsifying machine for 12 min at a rotation speed of 1500 rpm to obtain an initial emulsion. The pH value of the initial emulsion was adjusted to 6.0 by sodium carbonate solution. The initial emulsion was further emulsified by a high-pressure homogenizer under a pressure of 20000 psi. The emulsion was diluted to 1000 ml with water for injection, filtrated by 0.45 μm micro-porous filter membrane, the filtrate was separately packaged, charged with nitrogen, capped and sterilized by a rotary high-pressure steam sterilizer at 121° C. for 20 min to obtain the emulsion. The average particle size of the emulsion micro-particles was measured to be 168 nm with the pH value of 5.80.

### EXAMPLE 2

### Preparing Docetaxel Preparation for Intravenous Administration

a) Preparing the Drug Solution: 3.0 g docetaxel was added to 100 ml PEG-300, and stirred at 70° C. to dissolve the docetaxel. The pH value of the solution was adjusted to 6.0 by using hydrochloric acid and sodium hydroxide, and 0.2 g activated carbon for injection use was added to perform adsorption at 25° C. for 30 min. Next, the solution was filtrated by 0.45 μm micro-porous filter membrane, separately packaged, sterilized by high-pressure steam at 115° C. for 30 min, and the drug solution was thus obtained;

b) Preparing the Emulsion

b-1) Preparing the Oil Phase: 200 g soybean oil for injection was heated to 70° C. in water bath, into which 12 g soybean phospholipid for injection was added and dissolved by stirring, and then 0.5 g tocopherol was added and stirred well to obtain the oil phase;

b-2) Preparing the Water Phase: 22.5 g glycerol and 10 g poloxamer 188 were added into 640 ml water for injection, and stirred at 70° C. to dissolve the glycerol and poloxamer to obtain the water phase;

b-3) Preparing the Emulsion: the oil phase was mixed with the water phase at 70° C., and followed by emulsification using shear emulsifying machine for 10 min at a rotation speed of 1000 rpm to obtain an initial emulsion. The pH value of the initial emulsion was adjusted to 6.0 by sodium carbonate solution. The initial emulsion was further emulsified by a high-pressure homogenizer under a pressure of 10000 psi. The emulsion was diluted to 1000 ml with water for injection, filtrated by 0.45 μm micro-porous filter membrane, the filtrate was separately packaged, charged with nitrogen, capped and sterilized by a rotary high-pressure steam sterilizer at 121° C. for 20 min to obtain the emulsion. The average particle size of the emulsion micro-particles measured to be 177 nm with the pH value of 5.77.

### EXAMPLE 3

### Preparing Paclitaxel Preparation for Intravenous Administration

a) Preparing the Drug Solution: 8.0 g paclitaxel was added to 100 ml anhydrous ethanol, and stirred at 55° C. to dissolve the paclitaxel. The pH value of the solution was adjusted to 4.5 by using hydrochloric acid, and 4.5 g activated carbon for injection use was added to perform adsorption at 45° C. for 60 min. Next, the solution was filtrated by 0.45 μm micro-porous filter membrane to remove carbon, then filtrated by 0.22 μm micro-porous filter membrane to remove bacteria, separately packaged under a sterile condition, and the drug solution was thus obtained;

b) Preparing the Emulsion

b-1) Preparing the Oil Phase: a mixture of 10 g elemene oil, 45 g monodecanoin, 58 g didecanoin, 47 g helianthus annuus seed oil and 20 g evening primrose oil was heated to 75° C. in water bath, into which 65 g soybean phospholipid for injection, 5 g glyceryl monooleate and 3 g cholic acid were added and dissolved by stirring, and then 3.0 g tocopherol was added and stirred well to obtain the oil phase;

b-2) Preparing the Water Phase: 50 g sorbitol and 16 g sodium etiolate were added into 590 ml water for injection and stirred at 75° C. to dissolve the sorbitol and sodium cholate to obtain the water phase;

b-3) Preparing the Emulsion: the oil phase was mixed with the water phase at 75° C., and followed by emulsification using shear emulsifying machine for 170 min at a rotation speed of 2300 rpm to obtain an initial emulsion. The initial emulsion was further emulsified by a high-pressure homogenizer under a pressure of 12000 psi. The emulsion was diluted to 1000 ml with water for injection, and the pH value of the initial emulsion was adjusted to 7.1 by sodium hydroxide solution. The solution was filtrated by sintered filter funnel, and the filtrate was separately packaged, charged with nitrogen, capped and sterilized by performing high-pressure sterilization at 121° C. for 20 min to obtain the emulsion. The average particle size of the emulsion micro-particles was measured to be 250.7 nm with the pH value of 6.80.

### EXAMPLE 4

### Preparing Docetaxel Preparation for Intravenous Administration

a) Preparing the Drug Solution: 0.1 g docetaxel was added into a mixed solvent of 60 ml propylene glycol and 40 ml PEG-200, and stirred at 95° C. to dissolve the docetaxel. The pH value of the solution was adjusted to 6.5 by using a proper amount of citric acid and sodium carbonate, and 1.5 g activated carbon for injection use was added to perform adsorption at 100° C. for 30 min. Next, the solution was filtrated by 0.45 μm micro-porous filter membrane, separately packaged, sterilized high-pressure steam at 117° C. for 55 min, and the drug solution was thus obtained;

b) Preparing the Emulsion

b-1) Preparing the Oil Phase: a mixture of 25 g soybean oil for injection and 1.2 g oleic acid was heated to 58° C. in water bath, into which 10 g yolk phospholipid for injection was added and dissolved by stirring, and then 1 g tocopherol was added and stirred well to obtain the oil phase;

b-2) Preparing the Water Phase: 22.5 g glycerol and 20 g poloxamer 188 were added into 820 ml water for injection, and stirred at 58° C. to dissolve the glycerol and poloxamer to obtain the water phase;

b-3) Preparing the Emulsion: the oil phase was mixed with the water phase at 58° C., and followed by emulsification using shear emulsifying machine for 22 min at a rotation speed of 750 rpm to obtain an initial emulsion. The initial emulsion was further emulsified by colloid mill. The emulsion was diluted to 1000 ml with water for injection, and the pH value of the initial emulsion was adjusted to 5.6 by citric acid solution. The solution was filtrated by sand filtrating bar, and the filtrate was separately packaged, charged with nitrogen, capped and sterilized by performing high-pressure steam sterilization at 105° C. for 45 min to obtain the emulsion. The average particle size of the emulsion micro-particles was measured to be 63.2 nm with the pH value of 5.20.

### EXAMPLE 5

### Preparing Paclitaxel Preparation for Intravenous Administration

a) Preparing the Drug Solution: 4.0 g paclitaxel was added to a mixed solvent of 95 ml PEG-300 and 5 ml water for injection, and stirred at 60° C. to dissolve the pactlitaxel. The pH value of the solution was adjusted to 5.8 by using malic acid, and 0.8 g activated carbon for injection use was added to perform adsorption at 30° C. for 115 min. Next, the solution was filtrated by 0.45 μm micro-porous filter membrane, separately packaged, sterilized by circulating steam at 100° C. for 30 min to obtain the drug solution;

b) Preparing the Emulsion

b-1) Preparing the Oil Phase: a mixture of 42 g fish oil, 60 g sesame oil, 2 g tridecanoin, 18 g safflower seed oil, 5 g octyl and decyl monoglyceride and 23 g octyl and decyl diglyceride was heated to 60° C. in water bath, and stirred until dissolution was achieved, 2.1 g tocopherol was added and stirred well to obtain the oil phase;

b-2) Preparing the Water Phase: 50 g soybean phospholipid, 22.5 g glycerol, 7 g sodium cholate and 3 g sodium oleate were added into 700 ml water for injection, and stirred at 60° C. to dissolve the materials to obtain the water phase;

b-3) Preparing the Emulsion: the oil phase was mixed with the water phase at 60° C., and followed by emulsification using shear emulsifying machine for 80 min. at a rotation speed of 1600 rpm to obtain an initial emulsion. The initial emulsion was further emulsified by a high-pressure homogenizer under a pressure of 10000 psi. The emulsion was diluted to 1000 ml with water for injection, and the pH value of the initial emulsion was adjusted to 6.7 by sodium carbonate solution. The solution was filtrated by sintered filter funnel, then filtrated by 0.22 μm micro-porous filter membrane to remove bacteria and the filtrate was separately packaged, charged with nitrogen, and capped to obtain the emulsion. The average particle size of the emulsion micro-particles measured to be 128 nm with the pH value of 6.42.

### EXAMPLE 6

### Preparing Docetaxel Preparation for Intravenous Administration

a) Preparing the Drug Solution: 5 g docetaxel was added to a mixed solvent of 10 ml PEG-600, 40 ml propylene glycol and 50 ml anhydrous ethanol, and stirred at 65° C. until dissolved. The pH value of the solution was adjusted to 5.7 by using a proper amount of acetic acid, and 4 g activated carbon for injection use was added to perform adsorption at 60° C. for 100 min. Next, the solution was filtrated by 0.45 μm micro-porous filter membrane, separately packaged, sterilized by high-pressure steam at 121° C. for 30 min to obtain the drug solution;

b) Preparing the Emulsion

b-1) Preparing the Oil Phase: 15 g octyl and decyl glycerate was heated to 55° C. in water bath, into which 7 g soybean phospholipid for injection was added and stirred until dissolved to obtain the oil phase;

b-2) Preparing the Water Phase: 9 g sodium chloride was added into 950 ml water for injection, and stirred at 55° C. to dissolve the sodium chloride to obtain the water phase;

b-3) Preparing the Emulsion: the oil phase was mixed with the water phase at 55° C., and followed by emulsification by shear emulsifying machine for 8 min at a rotation speed of 375 rpm to obtain an initial emulsion. The initial emulsion was further emulsified by ultrasound. The emulsion was diluted to 1000 ml with water for injection, and the pH value of the initial emulsion was adjusted to 4.5 by hydrochloric acid solution. The solution was filtrated by 0.22 μm micro-porous filter membrane, and the filtrate was separately packaged, charged with nitrogen, and capped to obtain the emulsion. The average particle size of the emulsion micro-particles was measured to be 86.3 nm with the pH value of 4.37.

### EXAMPLE 7

### Preparing Paclitaxel Preparation for Intravenous Administration

a) Preparing the Drug Solution: 0.5 g paclitaxel was added to a mixed solvent of 25 ml glycerol and 75 ml anhydrous ethanol, and stirred at 60° C. to dissolve the paclitaxel. The pH value of the solution was adjusted to 5.8 by using malic acid, and 0.8 g activated carbon for injection use was added to perform adsorption at 30° C. for 115 min. Next, the solution was filtrated by 0.45 μm micro-porous filter membrane to remove carbon, separately packaged, sterilized by circulating steam at 100° C. for 30 min to obtain drug solution;

b) Preparing the Emulsion

b-1) Preparing the Oil Phase: mixture of 120 g linseed oil and 130 g sea buckthorn oil was heated to 80° C. in water bath, stirred to dissolve, into which 3.5 g tocopherol and 35 g cholic acid were added and stirred to dissolve well to obtain the oil phase;

b-2) Preparing the Water Phase: 30 g poloxamer (F68) for injection and 50 g yolk phospholipid were added into 550 ml water for injection and stirred to dissolve, into which 35 g sorbitol and 15 g mannitol were added, and stirred at 80° C. to dissolve the materials to obtain the water phase;

b-3) Preparing the Emulsion: the oil phase was mixed with the water phase at 80° C., and followed by emulsification by shear emulsifying machine for 200 min at a rotation speed of 6000 rpm to obtain an initial emulsion. The initial emulsion was further emulsified by a high-pressure homogenizer under a pressure of 20000 psi. The pH value of the initial emulsion was adjusted to 8.5 by sodium hydroxide solution or hydrochloric acid solution and diluted to 1000 ml with water for injection. The solution was filtrated by 0.45 μm micro-porous filter membrane, and the filtrate was separately packaged, charged with nitrogen, capped, and sterilized by high-pressure steam at 115° C. for 30 min to obtain the emulsion. The average particle size of the emulsion micro-particles as measured to be 320 nm with the pH value of 8.10.

### EXAMPLE 8

### Preparing Docetaxel Preparation for Intravenous Administration

a) Preparing the Drug Solution: 0.5 g docetaxel was added to a mixed solvent of 90 ml PEG-400 and 10 ml anhydrous ethanol, and stirred at 55° C. to dissolve the docetaxel. The pH value of the solution was adjusted to 6.8 by using a proper amount of sodium hydroxide, and 1.0 g activated carbon for injection use was added to perform adsorption at 40° C. for 60 min. Next, the solution was filtrated by 0.45 μm micro-porous filter membrane, separately packaged, sterilized by circulating steam at 100° C. for 30 min to obtain the drug solution;

b) Preparing the Emulsion

b-1) Preparing the Oil Phase: a mixture of 15 g Ganoderma lucidum spores oil, 92 g corn oil, 90 g monooctanoin, 100 g dicaprylin and 100 g linseed oil was heated to 85° C. in water bath, into which 4.5 g tocopherol and 42 g oleic acid were added and stirred to mix well to obtain the oil phase;

b-2) Preparing the Water Phase: 50 g soybean phospholipid, 43 g poloxamer (F68) and 50 g glucose were added into 400 ml water for injection and stirred at 85° C. to dissolve the materials to obtain the water phase;

b-3) Preparing the Emulsion: the oil phase was mixed with the water phase at 85° C. followed by emulsification by shear emulsifying machine for 269 min at a rotation speed of 7200 rpm to obtain an initial emulsion. The initial emulsion was further emulsified by a high-pressure homogenizer under a pressure of 24700 psi. The pH value of the initial emulsion was adjusted to 9.0 by sodium hydroxide solution and hydrochloric acid solution and diluted to 1000 ml with water for injection. The solution was filtrated by 0.45 μm micro-porous filter membrane, and the filtrate was separately packaged, charged with nitrogen, capped, and sterilized by high-pressure steam at 115° C. for 30 min to obtain the emulsion. The average particle size of the emulsion micro-particles was measured to be 472.4 nm with the pH value of 8.54.

### EXAMPLE 9

### Preparing Paclitaxel Preparation for Intravenous Administration

a) Preparing the Drug Solution: 0.05 g paclitaxel was added to a mixed solvent of 80 ml PEG-300 and 20 ml propylene glycol, and stirred at 50° C. to dissolve the paclitaxel. The pH value of the solution was adjusted to 6.0 by using hydrochloric acid, and 0.2 g activated carbon for injection use was added to perform adsorption at 30° C. for 30 min. Next, the solution was filtrated by 0.45 μm micro-porous filter membrane, and then filtrated by 0.22 μm micro-porous filter membrane to remove bacteria, and separately packaged to obtain the drug solution;

b) Preparing the Emulsion

b-1) Preparing the Oil Phase: 100 g soybean oil for injection was heated to 60° C. in water bath, into which 12 g soybean phospholipid for injection and 0.1 g oleic acid were added, and stirred to dissolve well to obtain the oil phase;

b-2) Preparing the Water Phase: 50 g glucose was added into 740 ml water for injection and stirred to dissolve at 60° C. to obtain the water phase;

b-3) Preparing the Emulsion: the oil phase was mixed with the water phase at 60° C., and followed by emulsification by shear emulsifying machine for 20 min at a rotation speed of 5500 rpm to obtain an initial emulsion. The pH value of the initial emulsion was adjusted to 6.2 by using sodium hydroxide and hydrochloric acid solution. The initial emulsion was further emulsified by a high-pressure homogenizer under a pressure of 15000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam sterilizer at 105° C. for 45 min to obtain the emulsion. The average particle size of the emulsion micro-particles was measured to be 160 nm with the pH value of 6.0.

### EXAMPLE 10

### Preparing Docetaxel Preparation for Intravenous Administration

a) Preparing the Drug Solution: 2.0 g docetaxel was added to a nixed solvent of 50 ml PEG-400 and 50 ml propylene glycol, and stirred at 80° C. to dissolve the docetaxel. The pH value of the solution was adjusted to 6.5 by using hydrochloric acid, and 0.4 g activated carbon for injection use was added to perform adsorption at 30° C. for 60 min. Next, the solution was filtrated by 0.45 μm micro-porous filter membrane, separately packaged, and sterilized by high-pressure steam at 100° C. for 60 min to obtain the drug solution;

b) Preparing the Emulsion

b-1) Preparing the Oil Phase: 150 g soybean oil for injection was heated to 70° C. in water bath, into which 12 g yolk phospholipid for injection was added and stirred to dissolve well to obtain the oil phase;

b-2) Preparing the Water Phase: 22.5 g glycerol and 0.2 g sodium oleate were added into 700 ml water for injection and stirred at 70° C. to dissolve the glycerol and sodium oleate to obtain the water phase;

b-3) Preparing the Emulsion: the oil phase was mixed with the water phase at 70° C., and followed by emulsification by shear emulsifying machine for 20 min at a rotation speed of 4500 rpm to obtain an initial emulsion. The pH value of the initial emulsion was adjusted to 7.0 by using sodium hydroxide and hydrochloric acid solution. The initial emulsion was further emulsified by a high-pressure homogenizer under a pressure of 12000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam sterilizer at 100° C. for 60 min to obtain the emulsion. The average particle size of the emulsion micro-particles was measured to be 240 nm with the pH value of 7.0.

### EXAMPLE 11

### Preparing Paclitaxel Preparation for Intravenous Administration

a) Preparing the Drug Solution: 2.5 g paclitaxel was added to a nixed solvent of 80 ml PEG-400 and 20 ml anhydrous ethanol, and stirred at 80° C. to dissolve the paclitaxel. The pH value of the solution was adjusted to 5.2 by using hydrochloric acid, and 0.3 g activated carbon for injection use was added to perform adsorption at 40° C. for 20 min. Next, the solution was filtrated by 0.45 μm micro-porous filter membrane, separately packaged, and sterilized by high-pressure steam 115° C. for 45 min to obtain the drug solution;

b) Preparing the Emulsion

b-1) Preparing the Oil Phase: 200 g soybean oil for injection was heated to 80° C. in water bath, into which 12 g yolk phospholipid for injection, 0.2 g oleic acid and 0.5 g tocopherol were added and stirred to dissolve well to obtain the oil phase;

b-2) Preparing the Water Phase: 20 g poloxamer and 22.5 g glycerol were added into 650 ml water for injection and stirred at 80° C. to dissolve the poloxamer and glycerol to obtain the water phase;

b-3) Preparing the Emulsion: the oil phase was mixed with the water phase at 80° C., and followed by emulsification by shear emulsifying machine for 20 min at a rotation speed of 6000 rpm to obtain an initial emulsion. The pH value of the initial emulsion was adjusted to 7.8 by using sodium carbonate solution. The initial emulsion was further emulsified by a high-pressure homogenizer under a pressure of 20000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam sterilizer at 115° C. for 45 min to obtain the emulsion. The average particle size of the emulsion micro-particles was measured to be 220 nm with the pH value of 7.8.

### EXAMPLE 12

### Preparing Docetaxel Preparation for Intravenous Administration

a) Preparing the Drug Solution: 1.5 g docetaxel was added to a mixed solvent of 60 ml PEG-400, 35 ml propylene glycol and 5 ml water, and stirred at 70° C. to dissolve the materials. The pH value of the solution was adjusted to 4.8 by using hydrochloric acid, and 0.6 g activated carbon for injection use was added to perform adsorption at 45° C. for 20 min. Next, the solution was filtrated by 0.45 μm micro-porous filter membrane, separately packaged, and sterilized by high-pressure steam at 121° C. for 30 min to obtain the drug solution;

b) Preparing the Emulsion

b-1) Preparing the Oil Phase: 250 g soybean oil for injection was heated to 70° C. in water bath, into which 12 g soybean phospholipid for injection and 0.8 g tocopherol were added and stirred to dissolve well to obtain the oil phase;

b-2) Preparing the Water Phase: 10 g poloxamer, 0.2 g sodium oleate and 9 g sodium chloride were added into 550 ml water for injection and stirred at 70° C. to dissolve the materials to obtain the water phase;

b-3) Preparing the Emulsion: the oil phase was mixed with the water phase at 70° C., and followed by emulsification by shear emulsifying machine for 10 min at a rotation speed of 7000 rpm to obtain an initial emulsion. The pH value of the initial emulsion was adjusted to 8.8 by using sodium hydroxide solution. The initial emulsion was further emulsified by a high-pressure homogenizer under a pressure of 16000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam stream sterilizer at 121° C. for 30 min to obtain the emulsion. The average particle size of the emulsion micro-particles was measured to be 260 nm with the pH value of 8.5.

### EXAMPLE 13

### Preparing Paclitaxel Preparation for Intravenous Administration

a) Preparing the Drug Solution: 3.5 g paclitaxel was added to 100 ml PEG-200, and stirred at 70° C. to dissolve the paclitaxel. The pH value of the solution was adjusted to 6.2 by using hydrochloric acid and sodium hydroxide, and 1 g activated carbon for injection use was added to perform adsorption at 30° C. for 30 min. Next, the solution was filtrated by 0.45 μm micro-porous filter membrane, separately packaged, and sterilized by high-pressure steam at 105° C. for 45 min to obtain the drug solution;

b) Preparing the Emulsion

b-1) Preparing the Oil Phase: 300 g soybean oil for injection was heated to 70° C. in water bath, into which 12 g soybean phospholipid for injection was added and stirred to dissolve well to obtain the oil phase;

b-2) Preparing the Water Phase: 5 g poloxamer and 22.5 g glycerol were added into 600 ml water for injection and stirred at 80° C. to dissolve the poloxamer and glycerol to obtain the water phase;

b-3) Preparing the Emulsion: the oil phase was mixed with the water phase at 80° C., and followed by emulsification by shear emulsifying machine for 30 min at a rotation speed of 4000 rpm to obtain an initial emulsion. The pH value of the initial emulsion was adjusted to 8.2 by using sodium hydroxide solution. The initial emulsion was further emulsified by a high-pressure homogenizer under a pressure of 15000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam sterilizer at 105° C. for 45 min to obtain the emulsion. The average particle size of the emulsion micro-particles was measured to be 310 nm with the pH value of 8.1.

### EXAMPLE 14

### Preparing Docetaxel Preparation for Intravenous Administration

a) Preparing the Drug Solution: 3 g docetaxel was added to 100 ml PEG-300, and stirred at 70° C. to dissolve the docetaxel. The pH value of the solution was adjusted to 4.5 by using hydrochloric acid, and 0.15 g activated carbon for injection use was added to perform adsorption at 45° C. for 60 min. Next, the solution was filtrated by 0.45 μm micro-porous filter membrane, separately packaged, and sterilized by circulating steam at 100° C. for 60 min to obtain the drug solution;

b) Preparing the Emulsion

b-1) Preparing the Oil Phase: a mixture of 100 g soybean oil for injection and 100 g octyl and decyl glycerate was heated to 80° C. in water bath, into which 0.8 g tocopherol were added and stirred to dissolve to obtain the oil phase;

b-2) Preparing the Water Phase: 12 g soybean phospholipid for injection use, 10 g poloxamer, 0.2 g sodium oleate and 22.5 g glycerol were added into 550 ml water for injection and stirred at 80° C. to dissolve to obtain the water phase;

b-3) Preparing the Emulsion: the oil phase was mixed with the water phase at 80° C., and followed by emulsification by shear emulsifying machine for 25 min at a rotation speed of 8000 rpm to obtain an initial emulsion. The pH value of the initial emulsion was adjusted to 7.8 by using sodium hydroxide solution. The initial emulsion was further emulsified by a high-pressure homogenizer under a pressure of 16000 psi and diluted to 1000 ml with water for injection. The solution was filtrated by 0.45 μm micro-porous filter membrane, and the filtrate was separately packaged, charged with nitrogen, capped, and sterilized by circulating steam at 100° C. for 60 min to obtain the emulsion. The average particle size of the emulsion micro-particles was measured to be 280 nm with the pH value of 7.9.

### EXAMPLE 15

### Preparing Paclitaxel Preparation for Intravenous Administration

a) Preparing the Drug Solution: 4 g paclitaxel added to 100 ml PEG-400, and stirred. at 70° C. to dissolve the paclitaxel. The pH value of the solution was adjusted to 6.0 by using hydrochloric acid and sodium carbonate, and 0.3 g activated carbon for injection use added to perform dsorptic 25° C. for 45 min. Next, the solution was filtrated by 0.45 μm porous filter membrane, separately packaged, and sterilized by high-pressure steam at 121° C. for 30 min to obtain the drug solution;

b) Preparing the Emulsion

b-1) Preparing the Oil Phase: a mixture of 75 g soybean oil for injection and 75 g octyl and decyl monoglyceride was heated to 80° C. in water bath, into which 2 g tocopherol was added and stirred to dissolve well to obtain the oil phase;

b-2) Preparing the Water Phase: 12 g yolk phospholipid for injection, 5 g poloxamer and 22.5 g glycerol were added into 600 ml water for injection and stirred at 80° C. to dissolve to obtain the water phase;

b-3) Preparing the Emulsion: the oil phase was mixed with the water phase at 80° C., and followed by emulsification by shear emulsifying machine for 20 min at a rotation speed of 8000 rpm to obtain an initial emulsion. The pH value of the initial emulsion was adjusted to 6.8 by using sodium hydroxide and hydrochloric acid solution. The initial emulsion was further emulsified by a high-pressure homogenizer under a pressure of 11000 psi and diluted to 1000 ml with water for injection. The solution was filtrated by 0.45 μm micro-porous filter membrane, and the filtrate was separately packaged, charged with nitrogen, capped, and sterilized by a rotary high-pressure steam sterilizer at 121° C. for 30 min to obtain the emulsion. The average particle size of the emulsion micro-particles vas measured to be 320 nm with the pH value of 6.6.

### Stability Studies of the Paclitaxel Preparation for Intravenous Administration

1. Taking the preparation prepared in accordance with the method of example 1 as the example, the drug solution was mixed homogenously with the emulsion in the ratio of 1:25. The change of drug contents, particle sizes of the emulsion micro-particles and pH values of these preparations were detected at different time-points.

Method:

4 ml of the drug solution was added to 100 ml of the emulsion, and stirred well. The drug contents, particle sizes and pH values of the preparations were determined at different time-points by HPLC, a particle size analyzer and a pH meter, respectively. When determining the drug content at the different time-points, all of the tested samples were to be filtrated firstly by 0.22 μm micro-porous filter membrane so as to remove the precipitated drugs crystals, and then the drug contents were determined. The change in the drug content was used to judge whether the drug was precipitated or not. In addition, the average particle sizes and pH values were determined directly. The average results are summarized in Table 1.

TABLE 1
Results of stability studies of the paclitaxel preparation for intravenous
administration

| | | | | | | | |
|---|---|---|---|---|---|---|---|
| Time (h) | 0 | 6 | 12 | 24 | 36 | 48 | 60 |
| Drug content (%) | 100 | 100.2 | 101.5 | 99.8 | 100.7 | 100.9 | 93.6 |
| Particle size (nm) | 168.7 | 175.0 | 171.5 | 167.8 | 170.0 | 176.4 | 297.0 |
| pH value | 5.71 | 5.64 | 5.77 | 5.80 | 5.77 | 5.65 | 5.66 |

2. Taking the preparation prepared in accordance with the method of example 1 as the example, the drug solution was mixed with the emulsion in the ratio of 1:5, shaken up homogenously, diluted with 10-fold volume of normal saline for injection and shaken up. The change of drug contents, particle sizes of the emulsion micro-particles and pH values of these preparations were detected at different time-points.

Method:

4 ml of the drug solution was added to 20 ml of the emulsion, and stirred well. Then the solution obtained was added into 200 ml normal saline for injection and shaken up homogenously. The drug contents, the particle sizes and pH values of the preparations were determined at different time-points by HPLC, a particle size analyzer and a pH meter. When determining the drug content at the different time, all of tested samples were to be filtrated firstly by 0.22 μm micro-porous filter membrane so as to remove the precipitated drugs crystals, and then the drug contents were determined. The change in the drug content was used to judge whether the drug was precipitated or not. In addition, the average particle sizes and pH values were determined directly. The average results are summarized in Table 2.

TABLE 2
Results of stability studies of the paclitaxel preparation for intravenous
administration

| | | | | | | | |
|---|---|---|---|---|---|---|---|
| Time (h) | 0 | 6 | 12 | 24 | 36 | 48 | 60 |
| Drug content (%) | 100 | 100.2 | 100.7 | 99.8 | 100.4 | 98.3 | 92.9 |
| Particle size (nm) | 177.5 | 178.2 | 180.8 | 178.4 | 187.9 | 192.4 | 222.7 |
| pH value | 5.27 | 5.24 | 5.37 | 5.18 | 5.22 | 5.31 | 5.12 |

It can be seen from Tables 1 and 2 that the drug contents in the filtrate of the paclitaxel preparation for intravenous administration are almost unchanged within 48 h, showing that no precipitation of paclitaxel has occurred. Besides this observation, the particle size and pH value did not show any significant change over time. All of these findings suggest that the paclitaxel preparations for intravenous administration were stable for at least 48 hours. At the 60^{th} hour, the drug content in the filtrate dropped slightly, indicating that a small amount of the drug was precipitated. Meanwhile, the particle size of the emulsion micro-particles was slightly increased; the change in the particle size also can be used to determine whether drug precipitation occurs or not. As shown in the above results, the preparations of the present invention are stable and comply with the requirements of clinical application.

What is claimed:

1. A drug solution of taxanes for intravenous administration, wherein the drug solution consists of paclitaxel or docetaxel at 0.01 to 10% (w/v), a pH regulator in an amount sufficient to adjust the pH of the drug solution to a pH of 4.0 to 7.0, with the balance of a solvent for injection,
wherein the solvent for injection is selected from one or more of PEG-200, PEG-300; PEG-400, PEG-600, propylene glycol, glycerol and anhydrous ethanol;
wherein the pH regulator is selected from one or more of citric acid, malic acid, hydrochloric acid, acetic acid, sodium carbonate, sodium bicarbonate and sodium hydroxide.

2. The drug solution of claim 1, wherein the pH regulator is selected from citric acid, malic acid, hydrochloric acid or sodium hydroxide.

3. The drug solution of claim 1, wherein:
the paclitaxel or the docetaxel being present at between about 0.01-5% (w/v); and
the pH value of the drug solution ranging from about 5.0 to 6.0.

4. The drug solution of claim 1, wherein in the drug solution, the content of the paclitaxel or the docetaxel is about 2.5% (w/v).

5. The drug solution of claim 4, wherein the solvent for injection is PEG-400.

6. The drug solution of claim 1, wherein the solvent for injection being selected from one or more of PEG-400, propylene glycol and glycerol.

7. A method for preparing a drug solution according to claim 1, the method comprising:
adding paclitaxel or docetaxel to a solvent for injection in a predetermined proportion and stirring at about 50-100° C. to dissolve, adjusting the pH value of the obtained solution to about 4.0-7.0 by using a pH regulator, adding activated carbon for injection use to perform adsorption, and taking the resulting solution and filtrating, separately packaging, sterilizing and packaging to obtain the drug solution.

8. The use of the drug solution of claim 1, characterized in that:
upon clinical administration, mixing the drug solution at the clinical dosage into an emulsion with no less than 5 times volume of the drug solution, and then adding an appropriate amount of normal saline or glucose solution for injection.

9. The use of claim 7, wherein the emulsion comprising at least one oil for injection at 1 to 50% (w/v), an emulsifier at 0.5 to 10% (w/v), an optional antioxidant at 0 to 0.5% (w/v), an isotonic regulator in an amount sufficient to adjust the osmotic pressure in the human body upon administration; an optional stabilizer at 0 to 5% (w/v), a regulator in an amount to sufficient adjust the pH of the emulsion to a pH of 4.0 to 9.0 with the balance of water for injection.

10. The use of claim 9, wherein the oil for injection comprises one or more of octyl and decyl glycerate, monooctanoin, dicaprylin, trioctanoin, Ganoderma lucidum spores oil, monodecanoin, didecanoin, tridecanoin, octyl and decyl monoglyceride, Brucea Javanica oil, coix seed oil, zedoary turmeric oil, Herba Artemisiae Annuae oil, octyl and decyl diglyceride, soybean oil, fish oil, linseed oil, helianthus annuus seed oil, evening primrose oil, sea buckthorn oil, safflower seed oil, sesame oil, corn oil, elemene oil and stearic acid.

11. The use of claim 9, wherein the emulsifier is one or more of soybean phospholipid, yolk phospholipid, cholesterol, poloxamer 188 and glyceryl monooleate.
